(19) United States
(12) Patent Application Publication
(10) Pub. No.: US 2010/0181220 A1
(43) Pub. Date: Jul. 22, 2010

(54) Shower Bag for Medical Devices

(75) Inventor: Sunil K. Dasara, Ann Arbor, MI (US)

Correspondence Address: TERUMO HEART, INC., 6200 JACKSON ROAD, ANN ARBOR, MI, 48103, US

(73) Assignee: TERUMO KABUSHIKI KAISHA, Tokyo (JP)

(21) Appl. No.: 12/357,571

(22) Filed: Jan. 22, 2009

(51) Int. Cl. A61B 19/02; B65D 33/06; B65D 33/16; B65D 30/00

(52) U.S. Cl. 206/438; 383/12; 383/42; 383/108

(57) ABSTRACT

A shower bag is provided for a medical device system having an implanted portion, an extracorporeal portion, and a cable extending between the implanted portion and the extracorporeal portion, wherein the extracorporeal portion is mounted in a carrying case wearable by the patient. A water-resistant pouch of the shower bag has an interior cavity extending between a top end and a bottom end, front and back exterior surfaces extending continuously over the top end, and an opening for inserting the carrying case into the interior cavity. An internal fastener suspends the carrying case within the interior cavity in a manner to permit the extracorporeal portion to remain in the carrying case. A carrying strap has first and second ends attached to the pouch proximate to the top end. A re-sealable fastener is mounted to the pouch disposed alongside an edge of the opening for closing the opening except for a cable passage that is proximate to the bottom end.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to circulatory support systems such as a left ventricular assist system, and, more specifically, to a shower bag for protecting a wearable component of the circulatory support system (while remaining connected to an implanted component of the system) from water during a patient's shower.

A heart pump system known as a left ventricular assist system (LVAS) is used for providing long term patient support with an implantable pump connected to an externally-worn pump control unit and batteries. The LVAS improves circulation throughout the body by assisting the left side of the heart in pumping blood in order to treat end-stage congestive heart failure. One such system is the DuraHeart™ LVAS system made by Terumo Heart, Inc., of Ann Arbor, Mich. The DuraHeart™ system employs a centrifugal pump with a magnetically levitated impeller to pump blood from the left ventricle to the aorta. The pump is electronically controlled to provide a flow rate from two to eight liters per minute, for example. The controller typically contains a display screen for showing system status. The controller may be connected to a hospital or main console to allow a physician or care giver to set-up, adjust, and monitor the controller and pump units.

In addition to the controller, the extracorporeal portion of the circulatory support system includes batteries or other power supply. The batteries and controller are needed on a full time basis, so they are typically worn externally by the patient. A control and communication cable is connected between the implanted pump and the electronic controller via an exit site in the patient's skin.

Various kinds of carrying devices such as backpacks and shoulder bags have been used for LVAS systems and other similar portable medical devices. A carrying case having respective pockets or receptacles for each of the controller and battery components is often used to strap or sling the extracorporeal portion of an LVAS system to the patient. Carrying systems attempt to maximize patient comfort and mobility to improve quality of life while using the implanted system.

After sufficient healing of the exit site occurs, it is desirable to allow the patient to shower. However, the controller and battery are sensitive to exposure to water. Parts of the extracorporeal portion of the system such as display screens, indicator lamps, and/or control push buttons, are intended to be accessible during normal usage. Making the carrying case waterproof in order to adapt it for use in a shower would be possible, but is undesirable because 1) it would make the screen, lamps, and buttons less accessible, and 2) it would be difficult to thoroughly dry the carrying case after a shower. Residual moisture could encourage growth of bacteria, leading to a risk of infection.

To avoid exposure of the carrying case to water, dedicated shower bags have been used wherein the controller and batteries are removed from the carrying case and are transferred to the shower bag. However, the extra handling of the components is inconvenient and gives rise to risks that a component could be dropped or damaged or that a push button could be accidentally pushed.

It has also been known to transfer the electrical components to waterproof bags that are supported away from the patient, e.g., by a rolling hanger or being placed on the floor. Thus, previous shower bags for such circulatory support systems have been inconvenient and fail to provide patient mobility and a compact size.

### SUMMARY OF THE INVENTION

The present invention provides a shower bag with improved usability and patient mobility. It avoids removal of the extracorporeal components from their regular carrying case, and provides easy installation of a shower bag that achieves very effective water protection. The shower bag can be used with any medical devices having a wearable electric component connected to an implanted component such as a Right Ventricle Assist Device (RVAD), a Total Artificial Heart (TAH), a Biventricular Assist Devices (BVAD), an Intra-aortic balloon pump (IABP), and so on.

In one aspect of the invention, a shower bag is provided for a patient using a medical device system having an implanted portion, an extracorporeal portion, and a cable extending between the implanted portion and the extracorporeal portion, wherein the extracorporeal portion is mounted in a carrying case wearable by the patient. A water-resistant pouch has an interior cavity extending between a top end and a bottom end, front and back exterior surfaces extending continuously over the top end, and an opening for inserting the carrying case into the interior cavity. An internal fastener suspends the carrying case within the interior cavity in a manner to permit the extracorporeal portion to remain in the carrying case. A carrying strap has first and second ends attached to the pouch proximate to the top end. A re-sealable fastener is mounted to the pouch disposed alongside an edge of the opening for closing the opening except for a cable passage that is proximate to the bottom end.

### BRIEF DESCRIPTION OF THE DRAWINGS

FIG. 1 is a perspective view showing the elements of a circulatory support system.

FIG. 2 is a perspective view of the extracorporeal portion of the circulatory support system in its carrying case.

FIG. 3 is a perspective view of a preferred embodiment of a shower bag prior to connection with the circulatory support system in the carrying case.

FIG. 4 shows the shower bag turned inside out for connection to the carrying case.

FIG. 5 shows the carrying case stowed inside the shower bag after the shower bag is restored to its right-side out configuration.

FIG. 6 is a close-up view of the bottom of the shower bag of FIG. 5 with the cable passing through the sealed opening at the bottom of the shower bag.

FIG. 7 is an isometric view of an alternative embodiment using an internal strap and a front flap.

FIG. 8 illustrates the closing of the internal strap of FIG. 7.

FIG. 9 illustrates the closing of the front flap of FIG. 7.

### DETAILED DESCRIPTION OF PREFERRED EMBODIMENTS

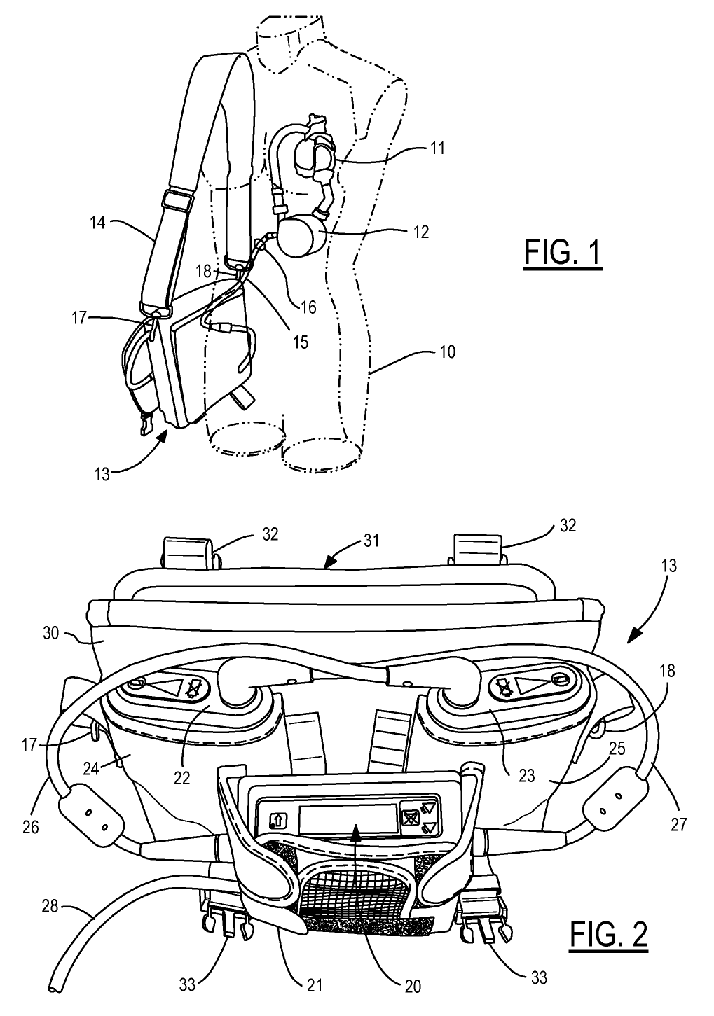

Referring now to FIG. 1, a torso of a patient 10 is shown in phantom with a patient's heart 11 connected to a circulatory support system including an implanted pump 12. The extracorporeal portion of the circulatory support system is contained in a carrying case 13 that can be slung on the patient by a shoulder strap 14. A control and communication cable or drive cable 15 extends between pump 12 and carrying case 13 via an exit site 16. Carrying case 13 typically includes separate sections or receptacles for the individual components joined together by a main housing or pack. Shoulder strap 14 may preferably be connected to the housing by loops, clips, or hooks, such as clips 17 and 18.

FIG. 2 shows carrying case 13 in greater detail. An electronic controller 20 is contained in a first pocket 21, while batteries 22 and 23 are retained in pockets 24 and 25, respectively. Battery cables 26 and 27 couple batteries 22 and 23 to controller 20, respectively. Communication/control cable 28 extends from controller 20 to the implanted pump (not shown).

Pockets 21, 24, and 25 are mounted to a base panel 30 which may include a fold-over front cover 31 (shown folded back in FIG. 2). Cover 31 has clips 32 that attach to clips 33 to hold cover 31 over the extracorporeal components for protection.

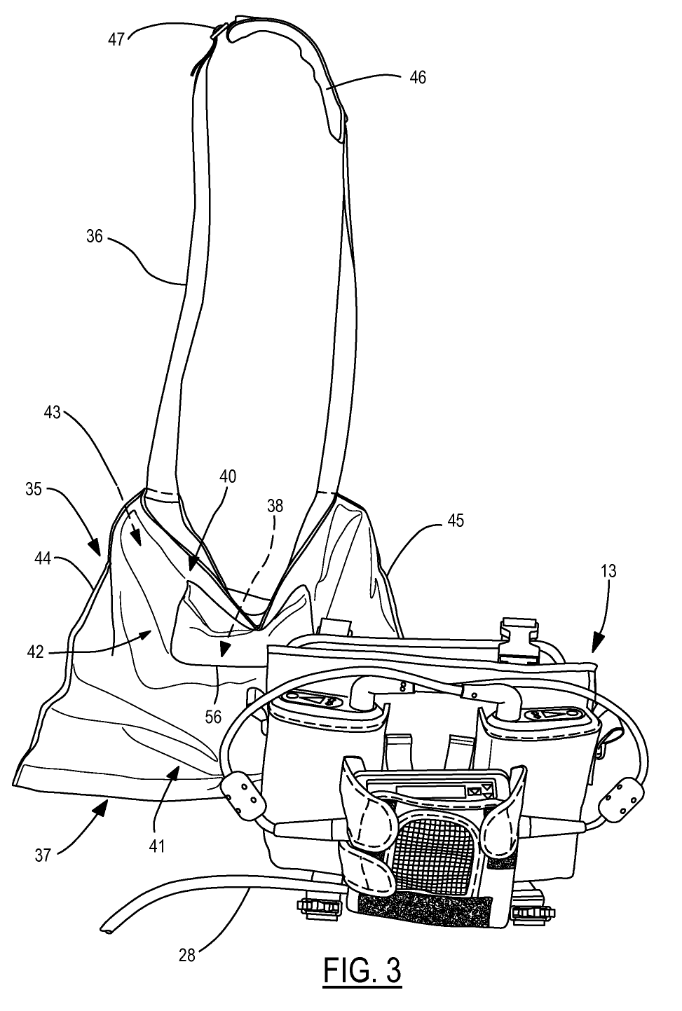

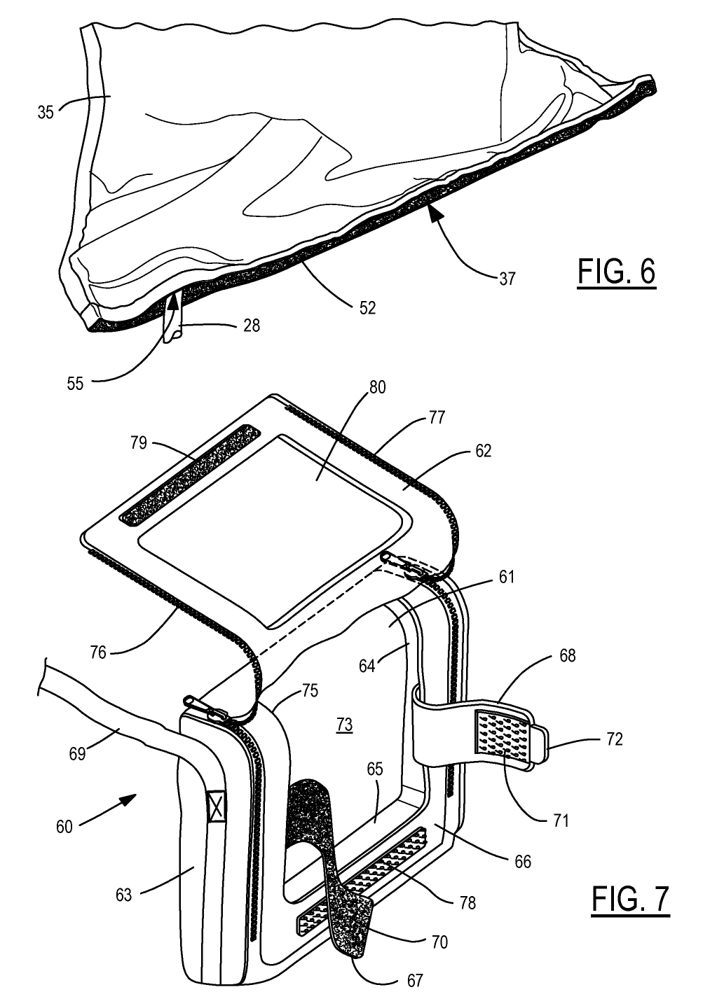

FIG. 3 shows carrying case 13 together with the shower bag of the present invention which employs a water resistant pouch 35 having an integral carrying strap 36. In a first preferred embodiment shown in FIGS. 3-6, pouch 35 has an opening 37 at the bottom end thereof for receiving carrying case 13 and the extracorporeal components. When pouch 35 is configured in its normal configuration (i.e., without the pouch being turned inside-out), an interior cavity 36 is present within pouch 35 extending between a top end 40 and a bottom end 41. A front exterior surface 42 and a back exterior surface 43 extend continuously over top end 40 and are joined at lateral sides 44 and 45 (e.g., at a seam) so that the only path into interior cavity 38 is upward through opening 37 at bottom end 41. Thus, shower water is prevented from reaching interior cavity 38. As shown in FIG. 1, carrying strap 36 may be a shoulder strap having a waterproof cushioning pad 46 and a length adjusting mechanism 47. If desired, the sealed seams on pouch 35 may be covered by a decorative ribbon which may be sewn on.

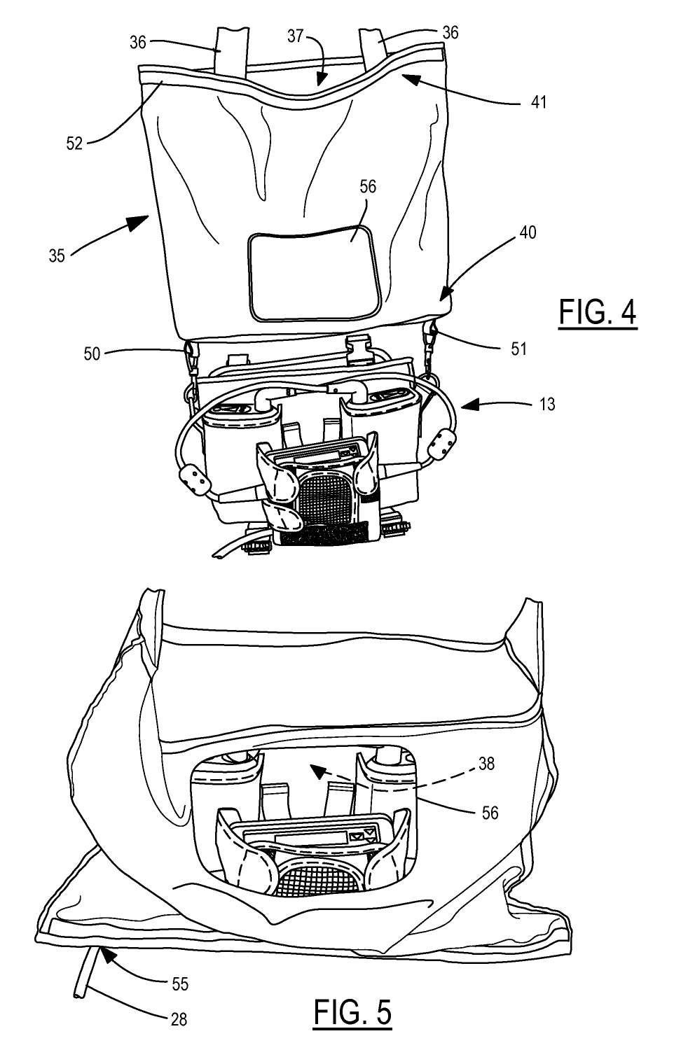

As shown in FIG. 4, pouch 35 can be turned inside-out to reveal internal fasteners 50 and 51 which may preferably take the form of clips (e.g., rotating carabiner latches) for attaching to attachment features 17 and 18 on carrying case 13. Thus, strap 14 would typically be removed from carrying case 13 in order to attach carrying case 13 to the shower bag, but carrying case 13 is connected to the shower bag without any removal or transfer of the system components. Preferably, carrying strap 36 may be supported by hanging it from a fixed structure when carrying case 13 is attached to internal fasteners 50 and 51. After joining fasteners 50 and 51 to carrying case 13, it is suspended by the shower bag. Next, pouch 35 is reconfigured to its normal configuration (e.g., by pulling pouch 35 downward over carrying case 13). The shower bag includes a re-sealable fastener such as strips of hook and loop fastener 52 disposed alongside an edge of opening 37. The opening is sealed except for a cable passage 55 that is proximate to bottom end 41. Thus, cable 28 exits the shower bag without any risk of kinking the cable. Cable passage 55 may result from a gap in the re-sealable fastener (e.g., if the hook and loop strips are shorter than the full length of the opening) or may simply be created by interfering with closure of the hook and loop fastener at the point where cable 28 exits.

In order to visualize the extracorporeal electronic components while in the shower bag, pouch 35 includes a transparent window 56 which may be formed of a lo clear plastic layer.

In the embodiment shown in FIGS. 3-6, pouch 35 creates a continuous layer around the top, front, back, left side, and right side of the pouch while the opening is disposed only at the bottom end. Since carrying case 13 is suspended within pouch 35, re-sealable fastener 52 does not bear any weight from the carrying case or extracorporeal components. Therefore, it can easily maintain a sufficient seal against ingress of water during the shower to protect the components.

FIGS. 7-9 show an alternative embodiment wherein a shower bag 60 includes a back panel 61 that transitions continuously into a front flap 62. Shower bag 60 further includes a left side panel 63, right side panel 64, bottom panel 65 and partial front 66. In this embodiment, the internal fastener for holding the carrying case is comprised of internal horizontal straps 67 and 68 that may be mounted to either back panel 61, side panels 63 and 64, or front panel 66. Straps 67 and 68 provide an adjustable tension to appropriately grip the carrying case. For example, strap 67 and 68 may include hook and loop fastener surfaces 70 and 71 with a pull tab 72 for tensioning the interlock during mounting and for peeling apart straps 67 and 68 during removal of the carrying case. A should strap 69 is attached to side panels 63 and 64 as shown.

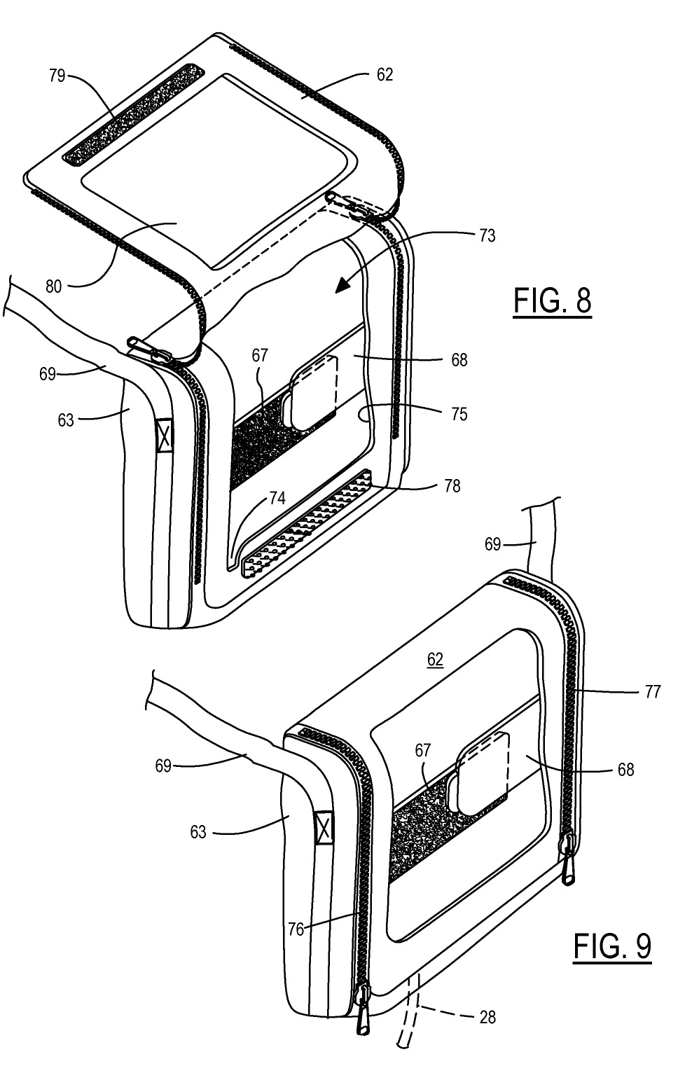

FIG. 8 shows straps 67 and 68 interconnected for gripping the carrying case (not shown) to suspend it within an interior cavity 73 of shower bag 60. FIG. 8 also shows an optional notch 74 within the opening 75 of partial front 66 for accommodating exit of the cable. FIG. 9 shows closing of front flap 62 using zipper closures 76 and 77 disposed along opposite lateral sides of flap 62, which results in a waterproof surface around the top, front, back, left side, and right side of shower bag 60. Additional hook and loop fasteners 78 and 79 may be disposed on partial front 78 and flap 79 for a re-sealable closing along the bottom edge of flap 62 in a manner that still allows the cable passage to be created at the bottom edge.

Front flap 62 includes a transparent window 80 which allows the extracorporeal circulatory support system components to be visually monitored during 10 the showering procedure.

Many conventional materials can be used to construct the shower bag of the present invention. Breathable waterproof materials are preferred for the pouch such as Ultrex and Goretex. For example, the pouch may be comprised of Derma-Plush fabric available from Brookwood Companies Incorporated of New York, N.Y. Most preferably, seams may be heat sealed using radio frequency heat sealing and the pouch fabric may comprise Derma-Plush RF Supreme. Various transparent, heat-sealable thermoplastics can be used for the window. In some embodiments, the carrying strap may be attached to a PVC tarpaulin extending at the top corners of the pouch to provide greater strength to support the internal fasteners. The tarpaulin is also heat sealed to the pouch in order to waterproof the holes. The carrying strap may be sewn to ears at the ends of the tarpaulin.

PVC plastic can also be employed for the clip fasteners, zippers, and rigid parts of the carrying strap and the internal fasteners, for example. Hook and loop fasteners of the type available under the trade name of Velcro can be used. The carrying strap and other straps incorporated into the fasteners may be a woven web material.

What is claimed is:

1. A shower bag for a medical device system having an implanted portion, an extracorporeal portion, and a cable extending between the implanted portion and the extracorporeal portion, wherein the extracorporeal portion is mounted in a carrying case wearable by a patient, the shower bag comprising:
a water-resistant pouch having an interior cavity extending between a top end and a bottom end, front and back exterior surfaces extending continuously over the top end, and an opening for inserting the carrying case into the interior cavity;
an internal fastener for suspending the carrying case within the interior cavity in a manner to permit the extracorporeal portion to remain in the carrying case;
a carrying strap having first and second ends attached to the pouch proximate the top end; and
a re-sealable fastener mounted to the pouch disposed alongside an edge of is the opening for closing the opening except for a cable passage that is proximate to the bottom end.

2. The shower bag of claim 1 wherein the internal fastener is comprised of at least one clip attached to the pouch in the interior cavity proximate the top end.

3. The shower bag of claim 1 wherein the exterior of the pouch includes a continuous layer around the top, front, back, left side, and right side of the pouch, and wherein the opening is disposed at the bottom end.

4. The shower bag of claim 3 wherein the internal fastener is comprised of a pair of clips attached to the pouch in the interior cavity proximate the top end, and wherein the pouch is adapted to be turned inside out so that the pair of clips are exposed for connection to the carrying case.

5. The shower bag of claim 3 wherein the re-sealable fastener is comprised of a hook and loop fastener mounted along the edge of the opening, and wherein the hook and loop fastener includes a gap for forming the cable passage.

6. The shower bag of claim 3 wherein the front exterior surface of the pouch includes a transparent section allowing visualization of the extracorporeal portion.

7. The shower bag of claim 1 wherein the internal fastener is comprised of lo a internal horizontal strap having an adjustable tension to grip the carrying case.

8. The shower bag of claim 7 wherein the front exterior surface includes a flap, and wherein the opening is covered by the flap.

9. The shower bag of claim 8 further including closures disposed along the lateral sides of the flap so that the closing of the closures results in a waterproof surface around the top, front, back, left side, and right side of the pouch.

10. The shower bag of claim 9 wherein the cable passage is located at a bottom edge of the flap.

11. The shower bag of claim 1 wherein the carrying strap has an adjustable length.

12. The shower bag of claim 1 wherein the implantable medical device system is a circulatory support system.
